(19) United States
(12) Patent Application Publication
(10) Pub. No.: US 2009/0004727 A1
(43) Pub. Date: Jan. 1, 2009

(54) METHOD FOR THE SEPARATION OF MICROORGANISMS ADHERED TO A SOLID SAMPLE BY MEANS OF A PHOSPHATE-BASED DAMPER AND SONICATION OF THE SAMPLE

(75) Inventors: Katia Nicole EHRENFELD STOLZENBACH, Nunoa (CL); Pilar Angelica PARADA VALDECANTOS, Nunoa (CL)

Correspondence Address: MERCHANT & GOULD PC, P.O. BOX 2903, MINNEAPOLIS, MN, 55402-0903, US

(73) Assignee: BIOSIGMA S.A., Colina (CL)

(21) Appl. No.: 12/163,212

(22) Filed: Jun. 27, 2008

(30) Foreign Application Priority Data
Jun. 29, 2007 (CL) 1926-2007

(51) Int. Cl. C12N 1/02

(52) U.S. Cl. 435/261

(57) ABSTRACT

The present invention provides a method for the separation of microorganisms of a microbiological community present in a solid sample. And once the microorganisms are isolated, they are used in microbiological techniques, or their nucleic acids, both DNA and RNA, are extracted from them for carrying out molecular biology techniques. In accordance with the present invention, the method for the separation of microorganisms adhered to a solid sample comprises the following stages: Providing a solid sample with microorganisms adhered to it; adding to the solid sample a high molarity phosphate-based buffer containing a phosphate solution of a concentration greater than 0.5 M, ethanol, and a non-ionic surfactant; sonicating, and recovering the supernatant, comprising microorganisms separated from the solid sample.

### SCOPE OF THE INVENTION

The present invention discloses a method for separating the microorganisms of a biological community present in a solid sample, using a high molarity-based buffer (above 0.5M) and the sonication technique, for this purpose.

### BACKGROUND INFORMATION OF THE INVENTION

The analysis of microorganisms associated with solid samples is highly relevant in various areas of biotechnology such as biomining, bioremediation, and in general wherever evaluating the characteristics of biological communities, whether wild or introduced, associated with a specific sample is required, This solid sample can for instance be a mining resource, understood as ore concentrates, fresh ores, pyrite, chalcopyrite, coveline, tailings, leach tailings, gangue, and metallic and non-metallic species in general; etc.

An area of the technique that is of great commercial interest is biomining. Biomining can be defined as the use of microorganisms in the recovery of metals from ores. Its most traditional expression is bioleaching, which is the solubilization of metals from complex matrixes in an acid medium, employing the direct or indirect action of microorganisms. But biomining is more than this process alone, it is also the monitoring and intervention of the microorganisms involved, because these techniques are complex and constantly developing. It is also the laboratory-level research associated with improvement of processes and development of new methodologies.

For bioleaching to develop effectively, it is convenient to control, and even intervene, the microbiological community that carries out the process. This control can be carried out by applying techniques with which it is possible to evaluate the microorganisms present in a mineral.

Normally, the analysis of a microbiological community can be conducted using various microbiological or molecular biology techniques. These techniques were designed for working with basically pure microorganism cultures not associated with non-biological materials. When these techniques are applied to complex environmental samples, very low performance is achieved, both because of the physical interference of ores present, and their oxide-reducing properties, that can damage the biological material required for the technique.

For example, one of the techniques most widespread in the detection and identification of microorganisms is the polymerase chain-reaction technique, PCR. This technique requires the presence of only a few intact nucleic acid molecules to start up the reaction. In spite of these minimal needs, it is extremely difficult to carry out a successful PCR amplification from samples extracted from a solid sample. This occurs because when the direct lysis method is used, cells are disrupted when they come into contact with their substrate, which in the case of mineral resources, has properties that are very aggressive with the nucleic acids released into the solution. Even when working with less aggressive ore substrates, such as soils or sediments, it is hard to obtain nucleic acids appropriate for being used as templates in the PCR technique (Miller et al, Appl. Environ. Microbiol. Vol 65: 4715-4724, 1999; Zhou et al, Appl. Environ. Microbiol. Vol 62: 316-322, 1996).

A second molecular biology approach to obtaining nucleic acids from mining resources, is to separate the cells present in a solid sample and subsequently disrupt these cells to purify the nucleic acids. In spite of the obvious advantages of this approach, because it removes the nucleic acids from the aggressive environment, there are relatively few publications in which this approach is used, and furthermore, they do not guarantee thorough recovery of the cells initially present in the sample.

The need to count on a method for removing microorganisms present in a solid sample persists in the technique, whether for using them in microbiological techniques which require that these separated microorganisms be viable, or for using them in molecular biology technologies, purifying the nucleic acids out of these microorganisms that have been separated from the ore.

With the present invention, this technical problem has been resolved, creating a method which separates the microbiological community from an ore with high concentrations of salts and metals to which it is adhered, using a high-molarity phosphate buffer and the sonication technique, obtaining whole and viable microorganisms which can be employed in traditional microbiological or molecular biology techniques.

It has been found that under certain controlled conditions when sonication is applied to a solid sample imbedded in a buffering solution, a supernatant containing whole microorganisms that are viable and can therefore be employed directly in microbiological techniques, or if preferred, in molecular biology techniques, seeing as nucleic acids can be easily extracted from these whole microorganisms, is obtained.

We have not found any studies in the-state-of-the-art that achieve successful separation of microorganisms present in a solid sample by means of the sonication technique, and specially not when these solids are mineral resources containing agents that are aggressive both towards live microorganisms and isolated DNA. There are some studies that refer to this technique for separating microorganisms present in soils or sediments, and even then, in conditions more “benign” than when working with mining resources, with not very favorable results. For example, Picard et al (Appl. Environ. Microbiol. Vol 58: 2717-2722, 1992) described sonication as the most efficient means of separating cells adhered to soils, but with the disadvantage that this sonication treatment that separates cells from soils leads to small DNA fragments. We believe that this fragmented DNA is the result of extreme sonication conditions without the appropriate buffer, in which cells are not only being loosened, but also destroyed. This does not occur with the method of the present invention, in which the cells that are separated from the solid sample are kept intact and viable, and, if the nucleic acids are purified they will be obtained in the same conditions as the ones obtained from a regular laboratory culture.

Another publication that approaches the sonication technique in soils is the one by Buesing and Gessner (Aquat. Microb. Ecol. Vol 27: 29-36, 2002). Four procedures for releasing bacteria associated with environmental sediment particles, using different instruments, are compared in this paper. The instruments evaluated were the rod sonicator with an intensity of 80 W; the bath sonicator with an intensity of 95 W; the Ultra-Turrax® tissue homogeniser and the Stomacher 80® laboratory blender. The samples were treated for 0.5 to 20 minutes in each case. It was found that the most efficient method for releasing bacteria from sediment was using a Stomacher 80®, and that extraction conditions using the other instruments are very severe, which leads to significant cell disruption. Buesing's results are not favourable for the sonication method. Nevertheless, because work was done with environmental samples with low concentrations of salts and metals, and that due to its design the Stomacher 80® grinding system adapts mainly to small-particle samples or paste, this system's efficiency with mining resource samples is debatable. These generally contain larger-size particles (2-5 cm diameter) and are rich in salts and minerals. Using the high-molarity phosphate buffer along with sonification presented in this patent, has proved to be efficient and flexible when processing mining resources from different sources with different particle sizes and concentrations of salts and minerals, and because of this it is presented as an effective alternative for resolving the problem of extracting cells from mineral resources.

On the other hand, Craig et al (Journal of Applied Microbiology, Vol 83: 557-565, 2002) evaluated a variety of techniques for separating bacteria from coastal sediments. Manual stirring; bath sonication, 700 W, 35 kHz for 6 and 10 minutes, and rod sonication, 100 W 20 kHz for 15 seconds and 1 minute, were compared. The 1-gram samples of sediment are mixed with 9 mL of peptonozed water (with 0.1% pepton) and undergo different treatments. The results show that bath sonication is more efficient in recovering bacteria from sediments with a high concentration of sand, whereas recovery lowers when the sediment contains slime, clay and organic carbon. In these last types of sediment, the most efficient method was manual stirring. The method developed in the present invention is different from the one described by Craig because it uses a high-molarity phosphate buffer, which permits a larger proportion of cells to be recovered and is of course more efficient than manual stirring.

As you can observe, no appropriate method for separating microorganisms from a solid sample with high concentrations of salts and metals has been described to date. The present invention will solve a problem of the technique, proving that it is possible to separate the microorganisms present in a solid sample using the sonication technique.

### SUMMARY OF THE INVENTION

The present invention discloses a method for separating microorganisms of a microbiological community present in a solid sample, and, once these microorganisms are isolated, using them in microbiological techniques, or extracting nucleic acids, both DNA and RNA, from them for carrying out molecular biology techniques.

The solid sample can for instance be a mineral resource containing for example, ore concentrates, fresh ores, pyrite, chalcopyrite, coveline, tailings, leach tailings, gangue, and in general, metallic and non-metallic species; etc. This method can be practices on any solid sample particle size, whether, in the case of an ore sample, just as obtained in the mine, crushed, ground or pulverized. It can likewise be applied to the biologically untreated solid sample or to one that has undergone culture, to increase the biomass present in it.

The method consists essentially in sonicating a solid sample embedded in a phosphate-based high molarity buffering solution in order to obtain a supernatant containing the whole microorganisms which can be employed in microbiological or molecular biology techniques.

The invention's sonication buffer includes phosphate buffer, ethanol, and a polysorbate 20 or 80 surfactant (Tween 20 (or Tween 80®). The buffer may additionally contain non-essential elements, such as anti-oxidizers, osmoregulators, chelators, lithic enzymes, solvents, and salts.

The mixture of solid sample and sonication buffer, is sonicated during a 15 to 300-second period, especially in a sonicating bath, especially during a 60 to 150-second period, preferably for 90 seconds. At a power level from 20 to 200 Watt, especially at power level from 80 to 150 Watt, more especially at 100 Watt, and at a frequency ranging from 10 to 100 KHz, especially from 25 to 75 KHz, more especially at 42 KHz.

Once sonication has been carried out, the biological material of interest is separated from the solid sample by traditional cell-separating methods. In which the cells obtained are viable, because of which they can be used in any microbiological or molecular biology method.

### BRIEF DESCRIPTION OF THE DRAWINGS

FIG. 1 shows the number of cells present in the culture (cellular count under the microscope) for the samples and controls on days 0, 1, 7 and 11 after treatment with the sonication method. No cell growth is observed in the medium without inoculum, whereas there is significant cell growth in the samples that were treated with the ‘Inoculated ore’ 1 and ‘Inoculated ore 2’ invention method, similar to that observed in ‘Wenelen culture’ and ‘Sonicated Wenelen’ positive controls.

Conclusion. The cells extracted from the solid sample using the method of the invention are viable.

FIG. 2 shows a gel in which a comparison is made of the nucleic acids of the samples submitted to the method of the invention, in which ‘Inoculated ore 1’=1 ‘Inoculated ore 2’=2, and of the positive controls ‘Wenelen culture’=2 and ‘Sonicated Wenelen’=5. The 4 samples present the same bands, the uppermost one corresponding to genomic DNA and the two lower ones, to ribosomal RNA.

Conclusion. The cells extracted from the solid sample by the method of the invention are metabolically active, which is made evident by the presence of the bands corresponding to ribosomal RNA (23S and 16Sr RNA).

FIG. 3, shows a 0.9% agarose gel dyed with ethidium bromide, in which the band corresponding to total DNA extracted from solid samples with the method of the invention using an extended sonication buffer which, apart from carrying essential components (high-molarity phosphate solution, ethanol and non-ionic surfactant) carries other unessential components such as lithic enzymes, sodium acetate, DMSO, EDTA and glycerol, is shown in tracks 1 and 2. The same band of total DNA extracted from the solid samples is shown in tracks 3 and 4, using the method of the invention this time, but with a phosphate-based buffer which only carries 3 essential components.

Conclusion: There is no substantial difference in the output of DNA extraction when a sonication buffer amplified or simplified according to the present invention is used.

### DETAILED DESCRIPTION OF THE PREFERRED EMBODIMENT(S)

The sonication technique is the application of ultrasound for different purposes. It has been traditionally used for stirring the particles within a vessel undergoing sonication, which allows dissolution of certain substances in a solvent to accelerate, which is useful when physical stirring is not possible, like in the case of tubes used for nuclear magnetic resonance (NMR). It can also be used for providing the energy needed for carrying out a chemical reaction.

Traditional biological applications of sonication are the disrupture of a variety of biological materials such as cells and vesicles, and deactivation of enzymes. It is also used for loosening biological materials, such as microorganisms or tissues, adhered to the walls of a vessel.

Sonication is conducted two typical ways, with a sonicator bath in which water transfers sonic energy from the transductor to the sample, or with a rod sonicator in which a metal rod submerged in the sample applies sonic energy. Any of these techniques can be employed in the present invention. When rod sonication is applied, the power and frequency of the sonic energy applied must be reduced, as opposed to when a sonic bath is used, and the sample must be protected in an ice bath to dissipate the caloric energy generated and so avoid damage to the microorganisms due to high temperatures. With bath sonication, sonic energy is modulated by water, and because of this, higher power or frequencies can be applied without damaging the microorganisms present.

The method of the present invention is useful for separating the microbiological community adhered to a solid sample with high concentrations of salts and metals, obtaining whole, viable microorganisms that can be employed in traditional microbiological and molecular biology techniques.

Included among the solid samples with high concentrations of salts and metals, on which the method of the invention can be applied, are mining resources associated to copper deposits or to deposits where other commercially interesting metals such as pyrite, chalcopyrite, coveline, tailings, leach tailings, gangue, non-metallic species, etc, are found.

This method can be carried out on any solid sample particle size, whether, as in the case of mineral resources, just as obtained in the mine, crushed, ground, or pulverized. It can likewise be applied on the biologically untreated solid sample or on the sample that has undergone culture, to increase the biomass present in it.

In the first stage, a solid sample from which the microorganisms present will be separated, is taken. An appropriate sample mass is 1 to 50 g, especially 5 to 20 g, preferably 10 g. The sample obtained is placed in an appropriate reaction tube, for instance a 50-mL Falcon tube.

A volume from the sonication buffer of the present invention (equivalent to the volume of the solid sample) is added over the volume of the solid sample. The sonication buffer includes:
Buffer based on a phosphate solution
ethanol
non-ionic surfactant

The phosphate solution can be within the pH range of 4 to 9, although it is equally efficient in any pH range, seeing as what is fundamental to the invention is the phosphate's high molarity rather than the buffer's pH. The phosphate buffer molarity can be over 0.5 M, preferably between 0.8 M and 2 M and most preferably 1 M.

Ethanol is used with a concentration of 5% to 20% weight/weight absolute ethanol (hereafter all weight/weight percentages are simply expressed as ‘%’), preferably at a concentration between 8% and 12%, most preferably 10%.

In which the non-ionic surfactant is preferably a polysorbate 20 or 80 surfactant (Tween 20® or Tween 80®), added with a concentration ranging from 0.01 to 0.5%, preferably 0.02 to 0.1%, and most specially 0.05%.

Conveniently, the sonication buffer of the invention has other non-essential compounds which give advantageous properties to this buffer.

The buffer may contain lithic enzymes which could help disrupt the biofilm and the molecules' adhesion cells, to make their release into the supernatant easier. Lysozymes, peptidases, such as carboxypeptidase A, B, C, Y; aminopeptidase, trypsin, chymoripsin, pepsine, etc., are examples of lithic enzymes. Lithic enzymes can be at a concentration of 0.1 to 1 mg/mL, preferably, at a concentration raging from 0.3 to 0.8 mg/mL, more preferably 0.5 mg/mL.

Other unessential components are salts that increase buffer molarity, such as sodium acetate, potassium acetate, sodium or potassium citrate, sodium or potassium sucinate, for example. Molarity of the salts should be 20 to 200 mM, preferably at a concentration of 50 to 150 mM, more specially 100 mM. Osmolarity regulators such as saccharose, trehalose, manitole, bethaine, glycine, etc, can also be used.

The buffer can also contain antioxydants such as DMSO (dimethyl sulfoxide), which can be at a concentration from 1 to 10%, especially 5%; and chelators such as EDTA (ethylene diamine tetraacetic acid), EGTA (ethylene glycol tetraacetic acid) which can be at a molarity of 10 to 100 mM, preferably at 50 mM. Conveniently, the buffer also includes glycerol as a thickener, which can be at a concentration of 1 to 10%, especially at 5%.

It may be convenient that the buffer contain a reducing agent, which can for instance be chosen from among β-mercapto ethanol, DTT or glutathione.

In a preferred type, the sonication buffer includes
Buffer based on a phosphate solution
Ethanol
Polysorbate 20 or 80 surfactant (Tween 20® or Tween 80®)
lithic enzymes
sodium acetate
DMSO
EDTA
glycerol

The solid sample and sonication buffer mixture is sonicated during a 15 to 300-second period, preferably in a sonicating bath, especially during a 60 to 150-second period, and more specially during 90 seconds. At 50 to 200 Watt power, especially at 80 to 150 Watt power, more specially at 100 Watt power, and a frequency from 10 to 100 KHz, especially from 25 to 75 KHz, most specially at 42 KHz. In this stage the microorganisms associated to the solid sample come loose, remaining in suspension in the sonication solution.

In order to separate the supernatant with microorganisms from the solid sample, any method known in the technique for the separation of cells, can be used.

In a preferred performance the sonicated reaction tube can be submitted to cold centrifugation to avoid degradation of nucleic acids and favor decantation. Centrifugation should be carried out under moderate conditions so that the solid sample settles while the biological material remains in suspension. Centrifugation can be carried out for 15 to 10 minutes at 500 to 3.000 g power. Centrifugation is especially done at 1.000 g for 2 minutes. After centrifugation the supernatant containing the released cells is recovered.

In order to separate the released cells from the other components in suspension, the recovered supernatant can be centrifuged. This centrifugation should be carried out in conditions such that the cells remain in the pellet while the other smaller elements such as polysacharid organic residues, proteins, lipides, or simply salts, remain in the supernatant, which can later be discarded. It is convenient that centrifugation be carried out at 4° C., to avoid degradation of the biological material. In a preferred condition, centrifugation is carried out during 5 minutes at over 4.000 g in a centrifuge refrigerated at 4° C.

The pellet obtained can be treated like any cell lump, and its subsequent treatment depends on the microbiological or molecular biology technique that will be used. For example, the pellet obtained can be resuspended for use as inoculum for a culture, or for doing a cell count, or it can be used for isolating nucleic acids using any of the traditional techniques that exist in the technique and work with these nucleic acids on PCR techniques, DNA or RNA arrangements, FISH, sequencing, etc.

A buffering solution that can be used for resuspending the pellet for extraction of nucleic acids, is the STT tampon (TRIS 10 mM pH 8.0; EDTA 20 mM; SDS 2%; TWEEN 20 1%; TRITON X100 1%; Biotechniques 27, p1140-p1145; 1999) supplemented with Proteinaze-K, and with sodium acetate 3M at pH 5.2.

A preferred method for obtaining the nucleic acids is fhenol-chloroform-isoamylic alcohol extraction, well known to experts in the technique. They can also extracted by nucleic acid extraction columns.

An example of the carrying out of the present invention is included below.

### Example 1

Two 4-g ore samples were inoculated with 40 mL of a culture of the Wenelen strain, the property of BioSigma (DSM 16786), grown in a 9K Fe²⁺ medium (10% p/v solution) and were incubated for 24 hours. Medium excess is subsequently removed, so that the ore is left humid with the cells adhered to it.

Each ore sample treated this way was put in a 50 mL Falcon tube, and a volume of the following sonication buffer equivalent to the volume of the sample is added to it:

| Buffer based on a phosphate solution at pH 5.0 | 1 | M |
|---|---|---|
| Polysorbate 20 surfactant (Tween 20) | 0.05% | |
| Ethanol | 10% | |
| Lithic enzymes | 0.5 | mg/mL |
| Sodium acetate pH 5.2 | 100 | mM |
| DMSO | 5% | |
| EDTA | 50 | mM |

| | |
|---|---|
| glycerol | 5% |

The ore and buffer mixture was sonicated for 90 s in an Ultrasonic ultrasound Bath at 100 Watt power and 42 KHz fixed frequency. The microorganisms associated to the mineral are released in this stage and remain in suspension in the sonication solution.

The reaction tubes were centrifuged for 2 minutes at 1.000 g in a centrifuge refrigerated at 4° C. After centrifuging, the supernatant containing the microorganisms was recovered; the lump containing the ore, was discarded.

The supernatants were centrifuged for 5 minutes at 9.000 g in a centrifuge refrigerated at 4° C. In this centrifugation, the cells remain in the pellet, and other smaller elements of the sample such as membranes or salts remain in the supernatant, which was eliminated.

The pellets of extracted cells were resuspended in 1 mL of nuclease-free water, and tagged as “Inoculated ore 1” and “Inoculated ore 2”. 0.5 mL were taken from each of these suspended samples and inoculated into 5 mL of 9K Fe²⁺ medium. The other 0.5 mL were used directly for extracting nucleic acids using the protocol described by Kiu Choong Syn et al, 1999.

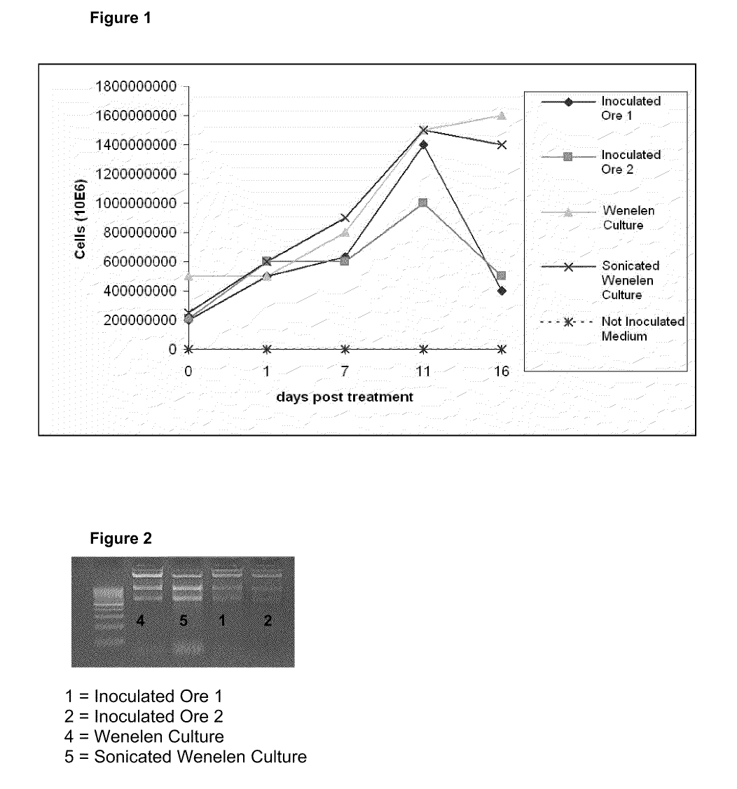

In order to evaluate the viability of cells obtained through the method of the invention, the samples in the culture medium were incubated for 11 days, and monitoring of the number of cells present in the culture over that period of time was done, as shown in FIG. 1.

Apart from the invention method samples, three controls were cultured:
Wenelen culture: 0.5 mL were taken from a grown Wenelen culture and inoculated into 5 mL of 9K Fe²⁺ medium. 0.5 mL were also taken to carry out an extraction of nucleic acids.
Sonicated Wenelen: 40 mL were taken from a grown Wenelen culture and sonicated under the same conditions as the tubes with ore, in other words, sonication buffer from the same invention was added, and sonicated over 90 s in an Ultrasonic ultrasound Bath at 100 Watt power and 42 KHz fixed frequency. It was subsequently centrifuged for 5 minutes at 9.000 g in a centrifuge refrigerated at 4° C. and the pellet obtained was resuspended in 1 mL of nuclease-free water. 0.5 mL were taken from this suspension to inoculate 5 mL of 9K Fe²⁺ medium, and 0.5 mL to carry out the extraction of nucleic acids.
Medium without inoculum: 0.5 mL of nuclease-free water was added to 5 mL of 9K Fe²⁺ medium.

The two samples, ‘Inoculated ore 1’ and ‘Inoculated ore 2’ and the three controls, ‘Wenelen culture’, ‘sonicated Wenelen’ and ‘Medium without inoculum’, were placed on a 6-well sterile plate which was incubated at 30° C., without stirring, over 11 days. 20-μl samples were taken from each well on days 0, 1, 7 and 11 with the purpose of counting the cells by observation under the microscope.

FIG. 1 shows microscopic cell count through phase contrast, for samples and controls on dates indicated after the treatment. It is observed that there is no cell growth in the medium without inoculum, whereas there is significant cell growth in the samples that are treated with the method of invention ‘Inoculated ore 1’ and ‘Inoculated ore 2’, similar to the one observed in positive controls ‘Wenelen culture’ and ‘Sonicated Wenelen’. This proves that the cells extracted from a solid sample using the method of the invention, are viable.

As it has been pointed out, nucleic acids were extracted using the protocol described by Kiu Choong Syn et al, 1999, using 5-mL aliquots from samples ‘Inoculated ore 1’, ‘Inoculated ore 2’, ‘Wenelen culture’ and ‘Sonicated Wenelen’. The nucleic acids extracted are shown in a gel in FIG. 2. It can be observed that the 4 samples present the same bands, of which the uppermost corresponds to genomic DNA, and the two lower ones to ribosomal RNA. The presence of the ribosomal bands is another clear indicator of the fact that the cells extracted from the solid sample by the method of the invention are viable and metabolically active.

### Example 2

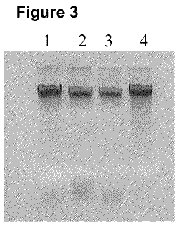

To evaluate the effectiveness of the method of separation of microorganisms from solid samples with a high molarity phosphate-based bufferer (sonication damper) with a simplified composition, versus an expanded composition, the following procedure was followed:
40 g of the same homogenized mineral sample was divided into four equal parts, to each of which a volume equivalent to 10 ml of high molarity phosphate-based damper was added.
In case A (replicas 1 and 2 of FIG. 3) an amplified composition sonication buffer was used:

| phosphate solution-based buffer at pH 5.0 | 1 | M |
|---|---|---|
| Polysorbate 20 surfactant (Tween 20) | 0.05% | |
| Ethanol | 10% | |
| Lithic enzymes | 0.5 | mg/mL |
| Sodium acetate pH 5.2 | 100 | mM |
| DMSO | 5% | |
| EDTA | 50 | mM |

| | |
|---|---|
| glycerol | 5% |

In the case of B (replicas 3 and 4) a simplified composition sonication buffer was used:

| Phosphate solution-based buffer at pH 5.0 | 1 M |
|---|---|
| Polysorbate 20 surfactant (Tween 20) | 0.05% |
| Ethanol | 10% |

Both A and B ore and buffer mixtures were sonicated for 90 s in an Utrasonic ultrasound Bath at 100 Watts of power and a fixed frequency of 42 KHz.
The four sonicated tubes were centrifuged during 2 minutes at 1,000 g in a centrifuge refrigerated to 4° C. Following centrifugation, the supernatant containing microorganisms was recovered; the pellet containing the ore was discarded.
The supernatants were centrifuged during 5 minutes at 9.000 g in a centrifuge refrigerated to 4° C. In this centrifugation the cells remained in the pellet, and other smaller elements in the sample, such as membranes or salts, remained in the supernatant, which was eliminated.
The extracted cell pellets were re-suspended in 1 mL of nuclease-free water. 0.5 ml were taken from this sample quantity and treated according to the protocol for the extraction of nucleic acids as described by Kiu Choong Syn et al, 1999.

FIG. 3 shows an agarose gel dyed with 0.9% ethidium bromide, showing that there is no substantial difference between using an extended or simplified sonication buffer to perform DNA extraction. Moreover, when the DNA extracted from the 4 replicas is quantified by a NanoDrop ND 1000 spectrophotometer, as shown in Table 1, the difference in concentration between 1 and 2 which were treated with the same type of sonicator is greater than the difference between 1 and 3 which were treated with different sonication buffers. The DNA concentration average of case A (1 and 2), 555.95 ng/ul, is very similar to the DNA concentration average of case B (3 and 4), 522.98 ng/ul. In consequence, it is concluded that both compositions are equally effective buffers, and that only in cases where the samples are too complex to treat is it advisable to add some or all of the optional components of the buffer (EDTA or EGTA, glycerol, Ithic enzymes, salts such as sodium acetate, potassium acetate, sodium or potassium citrate, potassium or sodium succinate, osmolarity regulators, DMSO and/or a reducing agent).

TABLE 1

| | Sample Identification | ng/ul |
|---|---|---|
| 1 | Sonication Buffer DNA | 43042 |
| | Amplified 1 | |
| 2 | Sonication Buffer DNA | 68148 |
| | Amplified 2 | |
| 3 | Sonication Buffer DNA | 45648 |
| | Simplified 1 | |
| 4 | Sonication Buffer DNA | 58949 |
| | Simplified 1 | |

What is claimed is:

1. A method for the separation of microorganisms adhered to a solid sample, wherein includes the following stages:
a. providing a solid sample with microorganisms adhered to it;
b. adding to the solid sample a high molarity phosphate-based buffer containing a phosphate solution of a concentration greater than 0.5 M, ethanol, and a non-ionic surfactant,
c. sonicating,
d. recovering the supernatant comprising the microorganisms separated from the solid sample.

2. A method for the separation of microorganisms adhered to the solid sample according to claim 1, wherein the high molarity phosphate-based buffer is at a pH between 4 and 9.

3. A method for the separation of microorganisms adhered to the solid sample according to claim 1, wherein ethanol is at a concentration of 5 to 20% absolute ethanol.

4. A method for the separation of microorganisms adhered to the solid sample according to claim 1, wherein the non-ionic surfactant is chosen from between a polysorbate 20 surfactant or a polysorbate 80 surfactant, and used at a concentration in the range of 0.01 to 0.5%; 0.02 to 0.1% preferably; and 0.05% more particularly.

5. A method for the separation of microorganisms adhered to the solid sample according to claim 1, wherein the high molarity phosphate-based buffer optionally includes other components chosen from among EDTA or EGTA, glycerol, lithic enzymes, salts such as sodium acetate, acetyl potassium, sodium or potassium citrate, sodium or potassium succinate, osmolarity regulators, DMSO and/or a reducing agent.

6. A method for the separation of microorganisms adhered to the solid sample according to claim 1, wherein sonicator bath sonication is carried out.

7. A method for the separation of microorganisms adhered to the solid sample according to claim 6, wherein sonication is performed for the duration of 15 to 300 seconds.

8. A method for the separation of microorganisms adhered to the solid sample according to claim 6, wherein sonication is performed for the duration of 60 to 150 seconds.

9. A method for the separation of microorganisms adhered to the solid sample according to claim 6, wherein sonication is performed at a power between 20 and 200 Watts.

10. A method for the separation of microorganisms adhered to the solid sample according to claim 6, wherein sonication is performed at a frequency between 10 and 100 kHz.

11. A method for the separation of microorganisms adhered to the solid sample according to claim 1, wherein the supernatant comprising microorganisms separated from the solid sample is separated from it by centrifugation.

12. A method for the separation of microorganisms adhered to the solid sample according to claim 1, wherein whole microorganisms are optionally separated from the other components present in the supernatant by centrifugation at more than 4,000 g, so that the cells remain in the pellet.

13. A method for the separation of microorganisms adhered to the solid sample according to claim 1, wherein microorganisms separated from the solid sample are used in microbiological or molecular biology techniques.

14. A method for the separation of microorganisms adhered to the solid sample according to claim 1, wherein the solid sample corresponds to mineral resources such as ore concentrates, fresh ores, pyrite, chalcopyrite, coveline, tailings, leach tailings, gangue, and metallic and non-metallic species in general.
